(19) United States
(12) Patent Application Publication
(10) Pub. No.: US 2024/0138907 A1
(43) Pub. Date: May 2, 2024

(54) MEDICAL DEVICE AND METHOD FOR FORMING SHUNT

(71) Applicant: TERUMO KABUSHIKI KAISHA, Tokyo (JP)

(72) Inventors: Tomoaki TAKEMURA, Kawasaki-shi (JP); Yusuke TAKAHASHI, Hadano-shi (JP)

(73) Assignee: TERUMO KABUSHIKI KAISHA, Tokyo (JP)

(21) Appl. No.: 18/398,992

(22) Filed: Dec. 28, 2023

Related U.S. Application Data
(63) Continuation of application No. PCT/JP2022/026999, filed on Jul. 7, 2022.

(30) Foreign Application Priority Data
Jul. 9, 2021 (JP) 2021-114254
Jul. 9, 2021 (JP) 2021-114256

(51) Int. Cl. A61B 18/14

(52) U.S. Cl. CPC A61B 18/1492; A61B 2017/00252

(57) ABSTRACT

A medical device and a method for forming a shunt configured to effectively cauterize a biological tissue having a variation in thickness. The medical device includes an expansion body, an elongated shaft portion to which the proximal end of the expansion body is fixed, a plurality of energy transfer elements disposed along the expansion body, and a pulling shaft are included, and the expansion body, which has a recess that defines a reception space, is configured such that an easy-to-deform portion that is disposed on the expansion body and is easily deformable is deformed to enlarge the reception space at the position corresponding to the easy-to-deform portion in the circumferential direction when a force in the axial direction of the expansion body is received.

### CROSS-REFERENCES TO RELATED APPLICATIONS

This application is a continuation of International Application No. PCT/JP2022/026999 filed on Jul. 7, 2022, which claims priority to Japanese Patent Application No. 2021-114254 filed on Jul. 9, 2021, and Japanese Patent Application No. 2021-114256 filed on Jul. 9, 2021, the entire content of all three of which is incorporated herein by reference.

### TECHNOLOGICAL FIELD

The present disclosure generally relates to a medical device including an expansion body that expands in a living body and a method for forming a shunt.

### BACKGROUND DISCUSSION

Chronic heart failure is a known heart disease. Chronic heart failure is broadly classified into a systolic heart failure and a diastolic heart failure on the basis of a cardiac function index. In a patient suffering from the diastolic heart failure, myocardial hypertrophy appears and stiffness (hardness) increases, whereby blood pressure increases in a left atrium and a cardiac pumping function is deteriorated. As a result, the patient may show heart failure symptoms such as a pulmonary edema. In addition, there is also a heart disease in which, due to pulmonary hypertension or the like, blood pressure increases on a right atrium side and the cardiac pumping function is deteriorated to exhibit heart failure symptoms.

In recent years, shunt treatments have attracted attention. For those patients who suffer from heart failure, a shunt (through-hole) serving as an escape route for increased atrial pressure is formed in an atrial septum, thereby helping alleviate heart failure symptoms. In the shunt treatment, the atrial septum is accessed using an intravenous approaching method, and the through-hole is formed to a desired size. Examples of a medical device for performing such a shunt treatment for the atrial septum include a device as disclosed in International Patent Application Publication No. WO 2020/094094 A.

In a medical device disclosed in International Patent Application Publication No. WO 2020/094094 A, a biological tissue is sandwiched between two expandable expansion bodies around an axis of an elongated shaft, and electrode portions, which are a plurality of energy transfer elements arranged in a circumferential direction of one of the expansion bodies, are brought into contact with the biological tissue to be arranged in a circumferential direction of a hole of the biological tissue to be treated, and then energy is applied from the plurality of electrode portions to cauterize the biological tissue. However, when thickness of the biological tissue varies in the circumferential direction of the expansion body, the electrode portions sandwiching a thin part of the biological tissue may be separated from the biological tissue. In addition, when the electrode portions are not sufficiently in contact with the tissue to be treated, sufficient energy may not be applied to the biological tissue, which may deteriorate the treatment effect.

### SUMMARY

A medical device and a method are disclosed for forming a shunt configured to effectively cauterize a biological tissue having a variation in thickness.

A medical device according to the present disclosure includes: an expansion body that has a distal end part including a force receiving portion and is expandable/contractible in a radial direction; an elongated shaft portion having a distal end part to which a proximal end of the expansion body is fixed; a plurality of energy transfer elements disposed along the expansion body; and a pulling shaft that is disposed inside the shaft portion, connectable to the force receiving portion of the expansion body by protruding from the distal end part of the shaft portion, and slidable with respect to the shaft portion, in which the expansion body includes: a first expansion portion having a distal-side expansion portion extending radially outward from the force receiving portion toward a direction of the proximal end and a distal-side top portion disposed on a proximal side of the distal-side expansion portion and convexly curved radially outward; a second expansion portion having a proximal-side expansion portion extending radially outward from the distal end part of the shaft portion toward a direction of the distal end and a proximal-side top portion disposed on a distal side of the proximal-side expansion portion and convexly curved radially outward; and a recess that is recessed radially inward, extends to couple the proximal-side top portion with the distal-side top portion, and defines a reception space configured to receive a biological tissue when the expansion body is expanded, the recess has a bottom portion located on an innermost side in the radial direction, a distal-side upright portion extending radially outward from a distal end of the bottom portion to the distal-side top portion, and a proximal-side upright portion extending radially outward from a proximal end of the bottom portion to the proximal-side top portion, one of the distal-side upright portion or the proximal-side upright portion includes a plurality of energy transfer element arrangement portions on which the plurality of individual energy transfer elements is disposed at substantially regular intervals in a circumferential direction of the expansion body, the other one of the distal-side upright portion or the proximal-side upright portion includes a plurality of facing portions facing the plurality of individual energy transfer elements when the expansion body is expanded, the distal-side expansion portion includes a plurality of distal-side strut structures coupled to the distal-side top portion, the proximal-side expansion portion includes a plurality of proximal-side strut structures coupled to the proximal-side top portion, at least one of the distal-side strut structures, the proximal-side strut structures, the energy transfer element arrangement portions, or the facing portions have easy-to-deform portions configured to be deformed more easily than other portions of the distal-side strut structures, the proximal-side strut structures, the energy transfer element arrangement portions, or the facing portions when a force in an axial direction of the expansion body is received, and each of the easy-to-deform portions deforms to enlarge the reception space at a position corresponding to the easy-to-deform portion in the circumferential direction.

A method for forming a shunt according to the present disclosure can form, in an oval fossa, a shunt through which a right atrium communicates with a left atrium using a medical device including an expansion body that has a distal end part including a force receiving portion and is expandable/contractible in a radial direction, an elongated shaft portion having a distal end part to which a proximal end of the expansion body is fixed, a plurality of energy transfer elements disposed along the expansion body, and a pulling shaft that is disposed inside the shaft portion, connectable to the force receiving portion of the expansion body by protruding from the distal end part of the shaft portion, and slidable with respect to the shaft portion, in which the expansion body includes a first expansion portion having a distal-side expansion portion extending radially outward from the force receiving portion toward a direction of the proximal end and a distal-side top portion disposed on a proximal side of the distal-side expansion portion and convexly curved radially outward, a second expansion portion having a proximal-side expansion portion extending radially outward from the distal end part of the shaft portion toward a direction of the distal end and a proximal-side top portion disposed on a distal side of the proximal-side expansion portion and convexly curved radially outward, and a recess that is recessed radially inward, extends to couple the proximal-side top portion with the distal-side top portion, and defines a reception space configured to receive a biological tissue when the expansion body is expanded, the recess has a bottom portion located on an innermost side in the radial direction, a distal-side upright portion extending radially outward from a distal end of the bottom portion to the distal-side top portion, and a proximal-side upright portion extending radially outward from a proximal end of the bottom portion to the proximal-side top portion, one of the distal-side upright portion or the proximal-side upright portion includes a plurality of energy transfer element arrangement portions on which the plurality of individual energy transfer elements is disposed at substantially regular intervals in a circumferential direction of the expansion body, the other one of the distal-side upright portion or the proximal-side upright portion includes a plurality of facing portions facing the plurality of individual energy transfer elements when the expansion body is expanded, the distal-side expansion portion includes a plurality of distal-side strut structures coupled to the distal-side top portion, the proximal-side expansion portion includes a plurality of proximal-side strut structures coupled to the proximal-side top portion, and at least one of the distal-side strut structures, the proximal-side strut structures, the energy transfer element arrangement portions, or the facing portions have easy-to-deform portions configured to be bent more easily than other portions of the distal-side strut structures, the proximal-side strut structures, the energy transfer element arrangement portions, or the facing portions when a force in an axial direction of the expansion body is received, the method including: inserting the medical device from an inferior vena cava into the right atrium; inserting the expansion body in a contracted state into a hole formed in the oval fossa; expanding the expansion body in the hole to dispose the biological tissue surrounding the hole in the reception space defined by the recess; sliding the pulling shaft in the direction of the proximal end with respect to the shaft portion to compress the expansion body such that the distal-side upright portion and the proximal-side upright portion of the recess approach each other; changing, according to thickness of the biological tissue surrounding the hole, a distance between the distal-side upright portion and the proximal-side upright portion in the circumferential direction of the expansion body on the basis of deformation of the easy-to-deform portion to bring the energy transfer elements disposed to face the recess along the distal-side upright portion or the proximal-side upright portion of the recess into contact with the biological tissue; and cauterizing the biological tissue disposed in the reception space using the energy transfer elements in contact with the biological tissue to inhibit occlusion due to natural healing of the hole.

According to the medical device and the method for forming a shunt configured as described above, the easy-to-deform portion deforms when the force in the axial direction acts on the expansion body so that the reception space at the position in the circumferential direction corresponding to the easy-to-deform portion enlarges. Thus, by deforming the easy-to-deform portion, it becomes possible to appropriately bring the plurality of energy transfer elements, which is arranged in the recess defining the reception space, into contact with the biological tissue having variations in thickness Therefore, the present medical device and the method for forming a shunt are enabled to rather effectively cauterize the biological tissue having variations in thickness.

The easy-to-deform portion may have bending rigidity lower than that of the other portions of the distal-side strut structure, the proximal-side strut structure, the energy transfer element arrangement portion, or the facing portion. With this arrangement, the force in the axial direction acts on the expansion body and the easy-to-deform portion is bent, whereby the reception space at the position in the circumferential direction corresponding to the easy-to-deform portion may be rather effectively enlarged.

The easy-to-deform portion may have an opening penetrating in the radial direction of the expansion body. With this arrangement, it becomes possible to relatively easily set the easy-to-deform portion, which can be easily bent, in the expansion body.

The easy-to-deform portion may have a thin portion having thickness in the radial direction of the expansion body smaller than that of an adjacent portion of the expansion body. With this arrangement, it becomes possible to relatively easily set the easy-to-deform portion, which can be easily bent, in the expansion body. Furthermore, it becomes possible to easily define the direction in which the easy-to-deform portion is bent.

The easy-to-deform portion may have a flexible portion made of a material more flexible than a material of an adjacent portion of the expansion body. With this arrangement, the bending rigidity of the easy-to-deform portion may be easily lowered.

The easy-to-deform portion may be sandwiched between rigid portions having the bending rigidity higher than that of the easy-to-deform portion in the axial direction of the expansion body. With this arrangement, it becomes possible to concentrate the stress on the easy-to-deform portion when the force in the axial direction acts on the expansion body so that the easy-to-deform portion may be easily bent.

The easy-to-deform portion may have a bent portion bent in a natural state. With this arrangement, it becomes possible to concentrate the stress on the bent portion when the force in the axial direction acts on the expansion body so that the easy-to-deform portion may be easily bent.

A medical device according to another aspect of the present disclosure includes: an expansion body that is expandable/contractible in a radial direction; an elongated shaft portion having, in a distal end part, a proximal-end fixing portion to which a proximal end of the expansion body is fixed; a pulling shaft that is disposed inside the shaft portion, connected to a distal end part of the expansion body by protruding from the distal end part of the shaft portion, and slidable with respect to the shaft portion; a distal-end shaft portion that extends inside the expansion body from a proximal end part to the distal end part of the expansion body; and an electrode portion disposed along the expansion body, in which the expansion body includes a recess that is recessed radially inward and defines a reception space configured to receive a biological tissue when the expansion body is expanded, the recess has a bottom portion located on an innermost side in the radial direction, a distal-side upright portion extending radially outward from a distal end of the bottom portion, and a proximal-side upright portion extending radially outward from a proximal end of the bottom portion, the electrode portion is disposed along the distal-side upright portion or the proximal-side upright portion to face the reception space, the pulling shaft is configured to apply, to the expansion body, a compressive force that makes compression along an axial center of the shaft portion such that the distal-side upright portion and the proximal-side upright portion approach each other by sliding in a direction of the proximal end with respect to the shaft portion, and in a state where the expansion body is expanded, the distal-end shaft portion includes a flexible portion configured to be bent at a center in an axial direction, a distal-end rigid portion disposed on a side distal of the flexible portion in the axial direction, and a proximal-end rigid portion disposed on a side proximal of the flexible portion in the axial direction.

A method for forming a shunt according to another aspect of the disclosure can form, in an oval fossa, a shunt through which a right atrium communicates with a left atrium using a medical device including an expansion body that is expandable/contractible in a radial direction, an elongated shaft portion having, in a distal end part, a proximal-end fixing portion to which a proximal end of the expansion body is fixed, a pulling shaft that is disposed inside the shaft portion, connected to a distal end part of the expansion body by protruding from the distal end part of the shaft portion, and slidable with respect to the shaft portion, a distal-end shaft portion that extends inside the expansion body from a proximal end part to the distal end part of the expansion body, and an electrode portion disposed along the expansion body, in which, in a state where the expansion body is expanded, the distal-end shaft portion includes a flexible portion configured to be bent at a center in an axial direction, a distal-end rigid portion disposed on a side distal of the flexible portion in the axial direction, and a proximal-end rigid portion disposed on a side proximal of the flexible portion in the axial direction, the method including: inserting the medical device from an inferior vena cava into the right atrium; inserting the expansion body in a contracted state into a hole formed in the oval fossa; expanding the expansion body in the hole to dispose a biological tissue surrounding the hole in a reception space defined by a recess of the expansion body having a bottom portion located on an innermost side in the radial direction, a distal-side upright portion extending radially outward from a distal end of the bottom portion, and a proximal-side upright portion extending radially outward from a proximal end of the bottom portion; compressing, by sliding the pulling shaft in a direction of the proximal end with respect to the shaft portion, the expansion body such that the distal-side upright portion and the proximal-side upright portion of the recess approach each other to bend the flexible portion according to thickness of the biological tissue surrounding the hole; bringing the electrode portion disposed to face the recess along the distal-side upright portion or the proximal-side upright portion of the recess into contact with the biological tissue by bending of the flexible portion; and cauterizing the biological tissue disposed in the reception space using the electrode portion in contact with the biological tissue to inhibit occlusion due to natural healing of the hole.

According to another aspect of the medical device and the method for forming a shunt configured as described above, it becomes possible to, when thickness of a biological tissue to be in contact with an expansion body varies along a circumferential direction, bend a distal-end shaft portion at a portion of a flexible portion depending on the thickness of the biological tissue to deform the expansion body such that a recess is brought into contact with each of portions of the biological tissue having larger and smaller thicknesses. As a result, it becomes possible to reliably bring the electrode portion into contact with the biological tissue over the entire circumference.

The distal-end rigid portion and the proximal-end rigid portion may be formed of an outer pipe into which the pulling shaft is inserted, and the flexible portion may be formed of a portion of the pulling shaft exposed from the distal-end rigid portion and the proximal-end rigid portion. With this arrangement, the rigidity of the distal-end rigid portion and the proximal-end rigid portion may be sufficiently secured.

The proximal-end rigid portion may be formed of an outer pipe into which the pulling shaft is inserted, and the pulling shaft may include the flexible portion exposed to the side distal of the proximal-end rigid portion in the axial direction, and the distal-end rigid portion disposed on the side distal of the flexible portion in the axial direction. With this arrangement, it becomes possible to reduce the number of outer pipes to facilitate assembly.

The distal-end rigid portion may be formed of an outer pipe into which the pulling shaft is inserted, and the pulling shaft may include the flexible portion exposed to the side proximal of the distal-end rigid portion in the axial direction, and the proximal-end rigid portion disposed on the side proximal of the flexible portion in the axial direction. With this arrangement, it becomes possible to reduce the number of outer pipes to facilitate assembly.

The distal-end shaft portion may be formed of an outer pipe into which the pulling shaft is inserted, and the distal-end shaft portion may include the flexible portion, the distal-end rigid portion, and the proximal-end rigid portion. With this arrangement, it becomes possible to reduce the number of outer pipes while eliminating the need to process the pulling shaft.

The pulling shaft may include the flexible portion, the distal-end rigid portion, and the proximal-end rigid portion. With this arrangement, it becomes possible to form the distal-end rigid portion and the proximal-end rigid portion only with the pulling shaft, whereby the number of parts may be further reduced.

### BRIEF DESCRIPTION OF THE DRAWINGS

FIG. 1 is a side view illustrating an overall configuration of a medical device according to a first embodiment.

FIG. 2 is an enlarged perspective view of a vicinity of an expansion body of the medical device.

FIG. 3 is a side view illustrating a distal end part of the medical device.

FIG. 4 is a front view of the medical device as viewed from a distal side.

FIG. 5 is a schematic view schematically illustrating a state in which the expansion body is disposed in a through-hole of an atrial septum.

FIG. 6 is a cross-sectional view illustrating a state in which a balloon is inserted into the atrial septum.

FIG. 7 is a cross-sectional view illustrating a state in which the distal end part of the medical device is inserted into the atrial septum.

FIG. 8 is a cross-sectional view illustrating a state in which the expansion body is disposed in the atrial septum.

FIG. 9 is a cross-sectional view illustrating a state in which a plurality of energy transfer elements disposed in a recess of the expansion body is brought into contact with a biological tissue.

FIG. 10 is a flowchart for explaining a method for forming a shunt.

FIGS. 11A to 11D are perspective views illustrating modified examples of the expansion body of the medical device according to the first embodiment, in which

FIG. 11A, FIG. 11B, FIG. 11C, and FIG. 11D illustrate a first modified example, a second modified example, a third modified example, and a fourth modified example, respectively.

FIG. 12 is an enlarged front view of a vicinity of an expansion body of a medical device according to a second embodiment.

FIG. 13 is an enlarged front view of a simplified expansion body of the medical device according to the second embodiment.

FIG. 14 is a view illustrating a state in which an electrode portion is pressed against a biological tissue when thickness of the biological tissue around a puncture hole varies in a circumferential direction, which is an enlarged view of the vicinity of the expansion body illustrating an atrial septum in a cross section.

FIG. 15 is a view illustrating a state in which, in a medical device having a distal-end shaft portion according to a fifth modified example of the second embodiment, an electrode portion is pressed against a biological tissue when thickness of the biological tissue around a puncture hole varies in a circumferential direction.

FIG. 16 is a view illustrating a state in which, in a medical device having a distal-end shaft portion according to a sixth modified example of the second embodiment, an electrode portion is pressed against a biological tissue when thickness of the biological tissue around a puncture hole varies in a circumferential direction.

FIG. 17 is a view illustrating a state in which, in a medical device having a distal-end shaft portion according to a seventh modified example of the second embodiment, an electrode portion is pressed against a biological tissue when thickness of the biological tissue around a puncture hole varies in a circumferential direction.

FIG. 18 is a view illustrating a state in which, in a medical device having a distal-end shaft portion according to an eighth modified example of the second embodiment, an electrode portion is pressed against a biological tissue when thickness of the biological tissue around a puncture hole varies in a circumferential direction.

FIG. 19 is a view illustrating a state in which, in a medical device according to a ninth modified example, an electrode portion is pressed against a biological tissue when thickness of the biological tissue around a puncture hole varies in a circumferential direction.

### DETAILED DESCRIPTION

Set forth below with reference to the accompanying drawings is a detailed description of embodiments of a medical device including an expansion body that expands in a living body and a method for forming a shunt. Note that dimensional ratios in the drawings may be exaggerated and different from actual ratios for convenience of description. In addition, in the present specification, a side of a medical device to be inserted into a living body lumen is referred to as a “distal side” and a side to be operated is referred to as a “proximal side”.

### First Embodiment

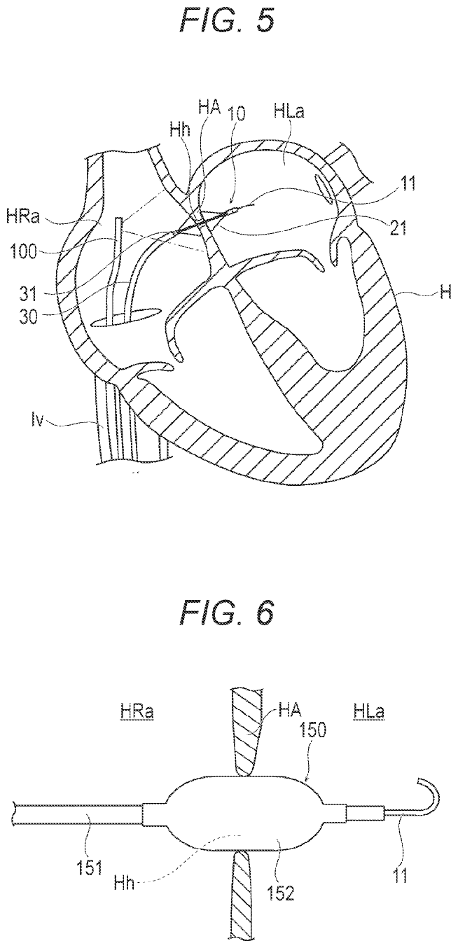

As illustrated in FIG. 5, a medical device 10 according to a first embodiment is configured to enlarge a through-hole Hh formed in an atrial septum HA of a heart H of a patient, and to further perform a maintenance treatment for maintaining the enlarged through-hole Hh at the increased size.

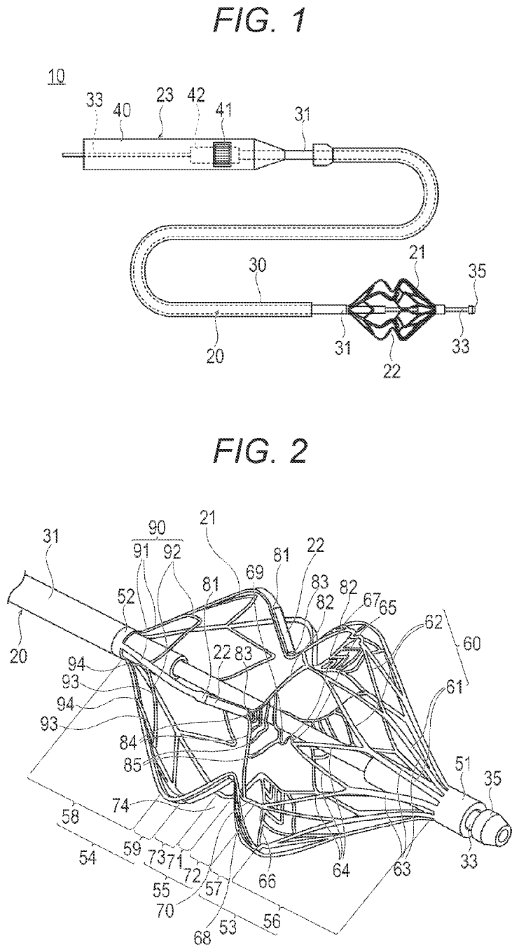

As illustrated in FIG. 1, the medical device 10 according to the first embodiment includes an elongated member 20 extending from a proximal end to a distal end, an expansion body 21 disposed on a distal end part of the elongated member 20, and an operation unit 23 connected to a proximal end part of the elongated member 20. An energy transfer element 22 (electrode portion) for performing the maintenance treatment described above is disposed on the expansion body 21.

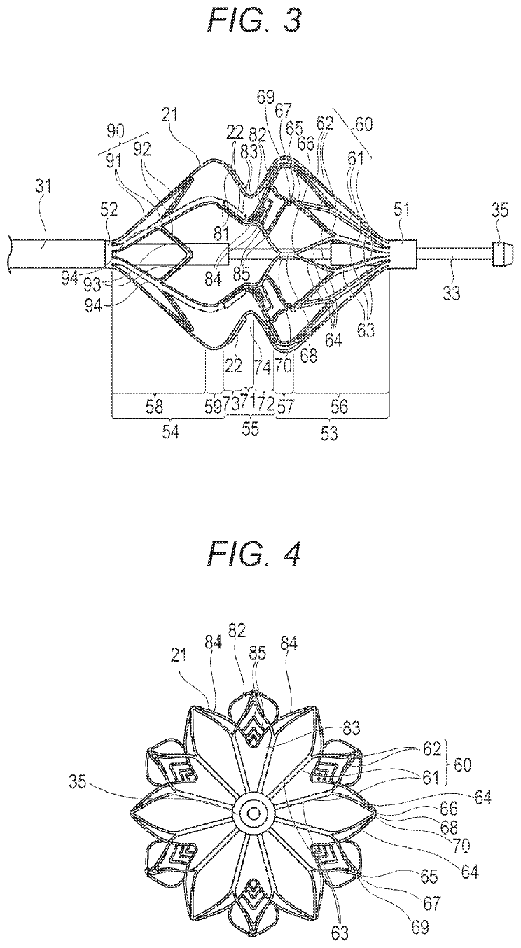

As illustrated in FIGS. 1 to 3, the elongated member 20 includes a shaft portion 31 holding the expansion body 21 at a distal end part, an outer tube 30 that accommodates the shaft portion 31, a pulling shaft 33, and a pulling portion 35 fixed to the distal end of the pulling shaft 33.

The shaft portion 31 is an elongated tubular body extending from the operation unit 23 to the expansion body 21. A proximal end part of the shaft portion 31 is fixed to a distal end part of the operation unit 23. A distal end part of the shaft portion 31 is fixed to a proximal end part of the expansion body 21.

The outer tube 30 is an elongated tubular body covering the shaft portion 31, and is movable forward and backward with respect to the shaft portion 31 in the axial direction (direction of the axial center of the elongated member 20). The outer tube 30 is configured to accommodate the contracted expansion body 21 in the outer tube 30 in a state of being moved to the distal side of the elongated member 20. With the outer tube 30 being moved to the proximal side from the state of accommodating the expansion body 21, the expansion body 21 may be exposed.

The pulling shaft 33 is an elongated tubular body disposed inside the shaft portion 31, and is movable forward and backward with respect to the shaft portion 31 in the axial direction. The pulling shaft 33 protrudes from the distal end of the shaft portion 31 toward the distal side, and protrudes from the distal end of the expansion body 21 toward the distal side. A distal end part of the pulling shaft 33 on a side distal of the expansion body 21 is fixed to the pulling portion 35. A proximal end part of the pulling shaft 33 is drawn out to a side proximal of the operation unit 23. A guide wire lumen is formed in the pulling shaft 33 along the axial direction, and a guide wire 11 (see FIGS. 5 to 7) may be inserted into the guide wire lumen.

The pulling portion 35 is an annular member fixed to an outer peripheral surface of a distal end part of the pulling shaft 33, and protrudes radially outward from the outer peripheral surface of the pulling shaft 33. The pulling portion 35 is not fixed to the expansion body 21. The outer diameter of the pulling portion 35 is larger than the inner diameter of the distal end part of the expansion body 21. Therefore, the pulling portion 35 is enabled to abut on the distal end part of the expansion body 21 from the distal side, pull the expansion body 21 toward the direction of the proximal end, and apply a compressive force for making compression along the axial direction of the shaft portion 31 to the expansion body 21.

The operation unit 23 can include a housing 40 to be gripped by an operator, a dial 41 configured to be operated by the operator, and a conversion mechanism 42 that converts rotation of the dial 41 into movement in the axial direction. The dial 41 is rotatably coupled to the housing 40. The dial 41 is partially exposed to the outside from an opening of the housing 40 so as to be operated by the operator. The pulling shaft 33 is held by the conversion mechanism 42 inside the operation unit 23. The conversion mechanism 42 can move the holding pulling shaft 33 forward and backward along the axial direction in conjunction with the rotation of the dial 41. For example, a rack and pinion mechanism may be used as the conversion mechanism 42.

As illustrated in FIGS. 2 to 4, the expansion body 21 includes a force receiving portion 51 disposed at the distal end of the expansion body 21, a proximal-end connecting portion 52 disposed at the proximal end of the expansion body 21, a first expansion portion 53 coupled to the force receiving portion 51, a second expansion portion 54 coupled to the proximal-end connecting portion 52, and a recess 55 disposed between the first expansion portion 53 and the second expansion portion 54.

The force receiving portion 51 may be annular, and is configured to receive a force directed toward the direction of the proximal end from the pulling portion 35 disposed on the distal side. The proximal-end connecting portion 52 may be annular, and is fixed to the distal end part of the shaft portion 31.

The first expansion portion 53 includes a distal-side expansion portion 56 extending radially outward from the force receiving portion 51 toward the direction of the proximal end, and a distal-side top portion 57 disposed on the proximal side of the distal-side expansion portion 56 and convexly curved radially outward.

The first expansion portion 53 includes a plurality of distal-side strut structures 60 extending radially outward from the force receiving portion 51 toward the direction of the proximal end and forming the distal-side expansion portion 56.

Each of the plurality of distal-side strut structures 60 includes a first section 61 extending from the force receiving portion 51 toward the direction of the proximal end, and a second section 62 extending from the proximal end of the first section 61 toward the direction of the proximal end and coupled to the distal-side top portion 57.

Each of the first sections 61 includes a first strut 63 extending from the force receiving portion 51 substantially parallel to the axial center of the expansion body 21 when viewed from the radial outside.

Each of the second sections 62 includes a plurality of second struts 64 bifurcated to spread in the circumferential direction of the expansion body 21 while extending from the proximal end of each of the first struts 63 toward the direction of the proximal end, and a first joint portion 65 and a second joint portion 66 coupled to the proximal end of the second strut 64. The first joint portion 65 and the second joint portion 66 are alternately arranged at substantially regular intervals in the circumferential direction of the expansion body 21 at the time of expansion. Each of the first joint portion 65 and the second joint portion 66 is formed such that two second struts 64, which are bifurcated from respective two first struts 63 arranged on the distal side and adjacent in the circumferential direction and are extending to approach each other, are joined together. The number of the first struts 63 disposed in the expansion body 21 can be, for example, 12, which is twice the number of the energy transfer elements 22. The number of the second struts 64 disposed in the expansion body 21 can be, for example, 24, which is twice the number of the first struts 63 and four times the number of the energy transfer elements 22. The number of the first struts 63 and the second struts 64 may be changed as appropriate.

Each of the first joint portions 65 is coupled to the distal-side top portion 57 disposed in the same phase as the energy transfer element 22 in the circumferential direction of the expansion body 21 with an auxiliary curved portion 67 that functions as a buffer portion interposed between the first joint portions 65 and the first distal-side top portion 69. The auxiliary curved portion 67 is curved in a wavelike shape to be folded a plurality of times when viewed from the radial outside.

Each of the second joint portions 66 is coupled to the distal-side top portion 57 disposed in a different phase in the circumferential direction of the expansion body 21 with respect to the energy transfer element 22 with a connecting strut 68 extending substantially parallel to the axial center of the expansion body 21 interposed between the second joint portions 66 and the second distal-side top portion 70 when viewed from the radial outside.

Each of the second struts 64 functions as an easy-to-deform portion that is more easily deformed than an adjacent portion on the distal side. The first strut 63 (rigid portion) having rigidity higher than that of the second strut 64 is disposed on the distal side of the second strut 64 (easy-to-deform portion).

The two second struts 64 are coupled to, via a first distal-side top portion 69, the distal side of the portion of the expansion body 21 on which the energy transfer element 22 is disposed. Those two second struts 64 are coupled to the two first struts 63 disposed on the distal side. Therefore, the sum of the rigidity of the two second struts 64, which serve as the easy-to-deform portion with rigidity K1, can be equal to or lower than the sum of the rigidity of the two first struts 63, which serve as the rigid portion with rigidity K2, and is preferably lower than the rigidity K2. In order to obtain such a second strut 64, the width of the second strut 64 (length of the expansion body 21 in the circumferential direction) is set to be smaller than the width of the first strut 63. The thickness of the second strut 64 (length of the expansion body 21 in the radial direction) may be set to be smaller than the thickness of the first strut 63.

In addition, the rigidity K1 of the easy-to-deform portion is preferably higher than rigidity K3 of the first distal-side top portion 69 supported by those two second struts 64, higher than rigidity K4 of one bottom connecting portion 83, and higher than rigidity K5 of one top portion included in a proximal-side top portion 59. This is because the bottom connecting portion 83, the first distal-side top portion 69, and the proximal-side top portion 59 need to be flexibly deformed for expansion of the expansion body 21.

Note that a position at which the easy-to-deform portion is disposed is not limited to the second strut 64 of the distal-side strut structure 60. The easy-to-deform portion may be disposed at a position other than the force receiving portion 51, the proximal-end connecting portion 52, the bottom portion 71, the distal-side top portion 57, and the proximal-side top portion 59 of the expansion body 21. Therefore, the easy-to-deform portion is disposed on at least one of the distal-side strut structure 60, a proximal-side strut structure 90, an energy transfer element arrangement portion 81, or a facing portion 82.

Note that a rigid portion having rigidity higher than that of the second strut 64 and the first distal-side top portion 69 may also be disposed between the second strut 64 (easy-to-deform portion) and the distal-side top portion 57. With this arrangement, the distal side and the proximal side of the second strut 64 are sandwiched between the rigid portions having rigidity higher than that of the second strut 64, thereby being relatively easily bent due to stress concentration.

The distal-side top portion 57 includes a plurality of first distal-side top portions 69 coupled to the auxiliary curved portion 67, and a plurality of second distal-side top portions 70 coupled to the connecting strut 68. The first distal-side top portions 69 and the second distal-side top portions 70 are alternately arranged at substantially regular intervals in the circumferential direction of the expansion body 21 at the time of expansion.

The recess 55 is recessed radially inward when the expansion body 21 is expanded, and extends to couple the proximal-side top portion 59 with the distal-side top portion 57. The recess 55 defines a reception space 74 configured to receive a biological tissue when the expansion body 21 is expanded.

The recess 55 includes the bottom portion 71 located on the innermost side in the radial direction, a distal-side upright portion 72 extending radially outward from the distal end of the bottom portion 71 to the distal-side top portion 57, and a proximal-side upright portion 73 extending radially outward from the proximal end of the bottom portion 71 to the proximal-side top portion 59.

The recess 55 includes a plurality of recessed strut structures 80 coupled to the plurality of distal-side strut structures 60 via the distal-side top portion 57. Each of the plurality of recessed strut structures 80 includes the energy transfer element arrangement portion 81 disposed on the proximal-side upright portion 73, and the facing portion 82 disposed on the distal-side upright portion 72, and also includes the bottom connecting portion 83 that couples a pair of the energy transfer element arrangement portion 81 and the facing portion 82 in the bottom portion 71. Each of the bottom connecting portions 83 is disposed in a phase different from that of the first strut 63 in the circumferential direction of the expansion body 21.

A plurality of the energy transfer element arrangement portions 81 is disposed at substantially regular intervals in the circumferential direction of the expansion body 21. The energy transfer element 22 is disposed on a surface of each of the energy transfer element arrangement portions 81 forming the inside of the recess 55.

The individual facing portions 82 face the individual energy transfer elements 22 when the expansion body 21 is expanded. Each of the facing portions 82 includes a plurality of distal-side upright struts 84 bifurcated to spread toward the direction of the distal end and a plurality of backrest portions 85 substantially along the circumferential direction of the expansion body 21 from the distal end of each of the bottom connecting portions 83. Each of the second distal-side top portions 70 is formed such that two distal-side upright struts 84, which are disposed on the proximal side and are extending to approach each other from the respective two bottom connecting portions 83 adjacent in the circumferential direction, are joined together. The plurality of backrest portions 85 couples the two distal-side upright struts 84 bifurcated from each of the bottom connecting portions 83. The plurality of backrest portions 85 is arranged side by side from the side closer to the bottom portion 71 to the side closer to the distal-side top portion 57. Each of the backrest portions 85 is curved such that a part between both ends coupled to the two distal-side upright struts 84 protrudes toward the distal-side top portion 57. Each of the backrest portions 85 is easily bent on the side closer to the distal-side top portion 57 with the both ends coupled to the distal-side upright struts 84 as supporting points. Therefore, the backrest portion 85 can be bent by a force toward the distal side received from the energy transfer element 22 disposed on the proximal-side upright portion 73. Accordingly, the biological tissue sandwiched between the energy transfer element 22 and the backrest portion 85 can be brought into contact with the energy transfer element 22. Among the plurality of backrest portions 85 forming each of the facing portions 82, the backrest portion 85 closest to the distal-side top portion 57 is coupled to the first distal-side top portion 69 at the part protruding toward the distal-side top portion 57. Note that the number of the backrest portions 85 forming each of the facing portions 82 is not particularly limited.

The second expansion portion 54 includes a proximal-side expansion portion 58 extending radially outward from the proximal-end connecting portion 52 toward the direction of the distal end, and the proximal-side top portion 59 disposed on the distal side of the proximal-side expansion portion 58 and convexly curved radially outward.

The proximal-side expansion portion 58 includes a plurality of proximal-side strut structures 90. Each of the proximal-side strut structures 90 is disposed in the same phase as the plurality of energy transfer element arrangement portions 81 in the circumferential direction of the expansion body 21. Each of the plurality of proximal-side strut structures 90 includes a plurality of third struts 91 extending from the distal end part of the shaft portion 31 to the proximal-side top portion 59 substantially parallel to the axial center of the expansion body 21 when viewed from the radial outside, and a plurality of secondary struts 92 coupling the third struts 91 adjacent in the circumferential direction. Each of the secondary struts 92 includes two support struts 93 joined to, at a junction 94, individual two third struts 91 adjacent in the circumferential direction. The two support struts 93 are coupled to have an angle between two junctions 94. Thus, each of the secondary struts 92 is formed to be longer than the linear distance between the two junctions 94. With this arrangement, even when the distance between the two junctions 94 increases at the time of expansion of the expansion body 21, the secondary strut 92 can continuously support the two third struts 91 while changing the angle between the two support struts 93 included in the secondary strut 92. Therefore, the expansion body 21 is enabled to expand by the compressive force applied by the pulling shaft 33 while expanding the third struts 91 at substantially regular intervals.

An interval between the proximal-side upright portion 73 and the distal-side upright portion 72 is preferably slightly larger in the axial direction on the outer side than the inner side in the radial direction when the expansion portion is expanded. With this arrangement, the biological tissue can be rather easily arranged between the proximal-side upright portion 73 and the distal-side upright portion 72 from the radial outside.

The energy transfer element 22 is disposed on a surface toward the distal side of the proximal-side upright portion 73 when the expansion portion is expanded. Since the energy transfer element 22 is disposed on the proximal-side upright portion 73, energy from the energy transfer element 22 is transmitted to the atrial septum HA from the right atrium side when the recess 55 sandwiches the atrial septum HA. In a case where the energy transfer element 22 is disposed on the distal-side upright portion 72, the energy from the energy transfer element 22 is transmitted to the atrial septum HA from the left atrium side.

The energy transfer element 22 can include, for example, a bipolar electrode that receives electric energy from an energy supply device, which is an external device. In this case, electricity is conducted between the energy transfer elements 22 disposed on individual arrangement portions of the energy transfer elements 22. The energy transfer element 22 and the energy supply device are connected to each other by a conductive wire coated with an insulating coating material. The conductive wire is drawn out (i.e., extends) to the outside via the elongated member 20 and the operation unit 23, and is connected to the energy supply device.

Alternatively, the energy transfer element 22 may be configured as a monopolar electrode. In this case, electricity is supplied from a counter electrode plate prepared outside a body. Furthermore, the energy transfer element 22 may be a heating element (electrode chip) that receives high-frequency electric energy from the energy supply device and generates heat. In this case, electricity is conducted between the energy transfer elements 22 disposed on individual wire rod portions. Moreover, the energy transfer element 22 may include an element configured to apply energy to the through-hole Hh, such as a heater including an electric wire or the like that provides heating and cooling operation or generates frictional heat by using microwave energy, ultrasound energy, coherent light such as laser, a heated fluid, a cooled fluid, or a chemical medium, and a specific form is not particularly limited.

While the energy transfer element 22 and the backrest portion 85 are disposed on the proximal-side upright portion 73 and the distal-side upright portion 72, respectively, in the present embodiment, the energy transfer element 22 and the backrest portion 85 may be disposed on the distal-side upright portion 72 and the proximal-side upright portion 73, respectively.

The expansion body 21 can be, for example, cut out from a pipe to be integrally formed. The struts forming the expansion body 21 may have a thickness, for example, in a range of 50 μm to 500 μm and a width, for example, in a range of 0.3 mm to 2.0 mm. However, the struts forming the expansion body 21 may have dimensions outside the ranges as set forth above. In addition, a shape of the struts is not particularly limited, and may be, for example, a circular cross-sectional shape or another cross-sectional shape.

The expansion body 21 may be formed of a metal material. Examples of the metal material that may be used include a titanium-based (Ti—Ni, Ti—Pd, Ti—Nb—Sn, etc.) alloy, a copper-based alloy, stainless steel, p-titanium steel, and a Co—Cr alloy. Note that an alloy having a spring property, such as a nickel titanium alloy, or the like may be more preferably used. However, a material of the wire rod portions is not limited to the above materials, and the wire rod portions may be formed of other materials.

The outer tube 30 and the shaft portion 31 of the elongated member 20 are preferably formed of a material having a certain degree of flexibility. Examples of such a material having a certain degree of flexibility of the outer tube 30 and the shaft portion 31 of the elongated member can include polyolefin such as polyethylene, polypropylene, polybutene, ethylene-propylene copolymer, ethylene-vinyl acetate copolymer, ionomer, or a mixture of two or more of them, soft polyvinyl chloride resin, polyamide, polyamide elastomer, polyester, polyester elastomer, polyurethane, fluorine resin such as polytetrafluoroethylene, polyimide, polyetheretherketone (PEEK), silicone rubber, and latex rubber.

The pulling shaft 33 and the pulling portion 35 may be formed of, for example, an elongated wire rod including a super elasticity alloy such as a nickel-titanium alloy and a copper-zinc alloy, a metal material such as stainless steel, a resin material having comparatively high rigidity, or the like. Furthermore, the pulling shaft 33 and the pulling portion 35 may be formed of the materials described above coated with a resin material such as polyvinyl chloride, polyethylene, polypropylene, ethylene-propylene copolymer, fluorine resin, or the like.

Next, a method for forming a shunt using the medical device 10 according to the first embodiment will be described with reference to a flowchart illustrated in FIG. 10. The present method for forming a shunt is performed on a patient suffering from heart failure (left heart failure). More specifically, as illustrated in FIG. 5, the present method is a treatment method to be performed on a patient suffering from chronic heart failure in which myocardial hypertrophy appears in a left ventricle of the heart H and stiffness (hardness) increases so that blood pressure increases in a left atrium HLa.

The treatment method according to the present embodiment includes forming the through-hole Hh in the atrial septum HA (S1), disposing the expansion body 21 in the through-hole Hh (S2), receiving a biological tissue in the reception space 74 (S3), enlarging the diameter of the through-hole Hh using the expansion body 21 (S4), confirming hemodynamics in the vicinity of the through-hole Hh (S5), performing the maintenance treatment for maintaining the size of the through-hole Hh (S6), and confirming the hemodynamics in the vicinity of the through-hole Hh after the maintenance treatment (S7).

At the time of forming the through-hole Hh, the operator delivers, to the vicinity of the atrial septum HA, an introducer in which a guiding sheath and a dilator are combined with each other. For example, the introducer may be delivered to a right atrium HRa via an inferior vena cava Iv. In addition, the introducer may be delivered using the guide wire 11. The operator may insert the guide wire 11 into the dilator and deliver the introducer along the guide wire 11. Note that the introducer and the guide wire 11 may be inserted into a living body using a method such as a method of using an introducer to be introduced into a blood vessel.

In the step of S1, the operator causes a puncture device to penetrate from the side of the right atrium HRa toward the side of the left atrium HLa to form the through-hole Hh. As the puncture device, a device such as a wire having a sharp distal end maybe used, for example. The puncture device is inserted into the dilator, and is delivered to the atrial septum HA. The puncture device may be delivered to the atrial septum HA instead of the guide wire 11 after the guide wire 11 is removed from the dilator.

Next, the operator delivers a balloon catheter 150 to the vicinity of the atrial septum HA along the guide wire 11 inserted in advance. As illustrated in FIG. 6, the balloon catheter 150 includes a balloon 152 at a distal end part of a shaft portion 151. When the balloon 152 is placed in the atrial septum HA, it is expanded in the radial direction to enlarge the through-hole Hh.

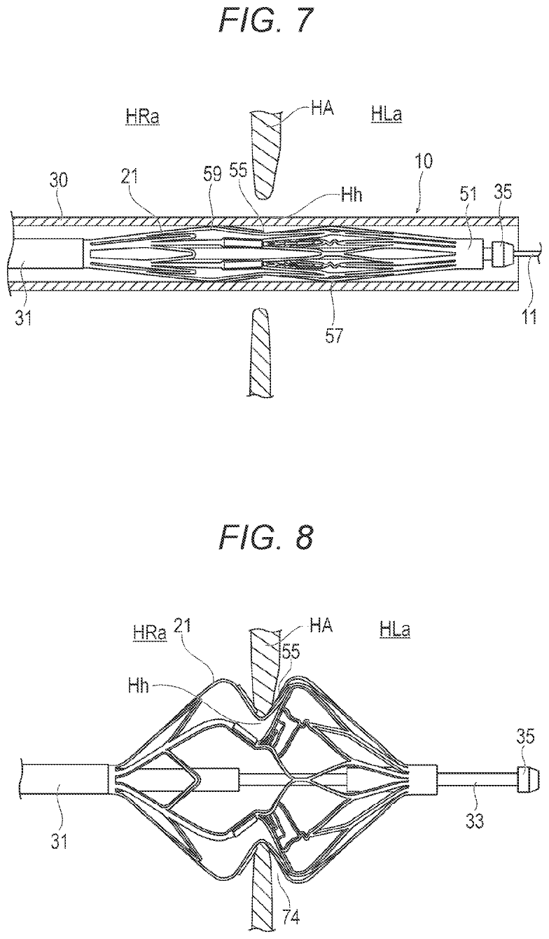

As illustrated in FIG. 7, in the step of S2, the medical device 10 is delivered to the vicinity of the atrial septum HA along the guide wire 11 inserted in advance. At this time, the distal end part of the medical device 10 penetrates the atrial septum HA and reaches the left atrium HLa. In addition, when the medical device 10 is inserted, the expansion body 21 is in a state of being housed in the outer tube 30.

Next, in the step of S3, the outer tube 30 is moved to the proximal side to expose the expansion body 21. As a result, as illustrated in FIG. 8, the diameter of the expansion body 21 increases and the recess 55 is arranged in the through-hole Hh of the atrial septum HA to receive the biological tissue surrounding the through-hole Hh in the reception space 74. The through-hole Hh is maintained in a state of being enlarged by the expansion body 21.

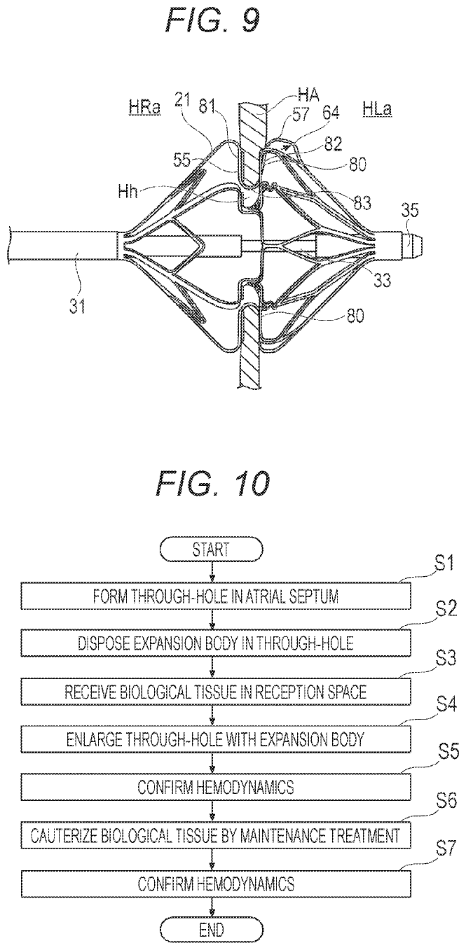

In the step of S4, the operator operates the operation unit 23 in the state where the atrial septum HA is received in the reception space 74 of the recess 55, moves the pulling shaft 33 to the proximal side, and sandwiches the biological tissue with the recess 55 of the expansion body 21, as illustrated in FIG. 9. Meanwhile, the thickness of the atrial septum HA (biological tissue) may be non-uniform in the circumferential direction. Each of the plurality of recessed strut structures 80 including the energy transfer element arrangement portion 81, the bottom connecting portion 83, and the facing portion 82 is independently deformable. In addition, the second strut 64 (easy-to-deform portion) is coupled to the distal side of each of the recessed strut structures 80 via the distal-side top portion 57. Thus, each of the recessed strut structures 80 may independently change the gap between the energy transfer element arrangement portion 81 and the facing portion 82 while deforming the second strut 64 depending on the thickness of the atrial septum HA sandwiched by the recessed strut structures 80. Therefore, the separation distance between the energy transfer element arrangement portion 81 and the facing portion 82 in the recessed strut structure 80 sandwiching the atrial septum HA partially thick in the circumferential direction is larger than the separation distance between the energy transfer element arrangement portion 81 and the facing portion 82 in the recessed strut structure 80 sandwiching the atrial septum HA partially thin in the circumferential direction. At this time, the second strut 64 coupled to, via the distal-side top portion 57, the distal side of the recessed strut structure 80 sandwiching the atrial septum HA partially thick in the circumferential direction is bent more than other second struts 64. Therefore, even when the thickness of the atrial septum HA is non-uniform in the circumferential direction, all the energy transfer elements 22 arranged in the recess 55 can be appropriately brought into contact with the atrial septum HA.

After the expansion body 21 is disposed in the through-hole Hh, the hemodynamics is checked in the step of S5. As illustrated in FIG. 5, the operator delivers a hemodynamics checking device 100 to the right atrium HRa via the inferior vena cava Iv. For example, an echo catheter may be used as the hemodynamics checking device 100. The operator can display an echo image obtained by the hemodynamics checking device 100 on a display device, such as a display, and can check blood volume passing through the through-hole Hh on the basis of a displayed result.

Next, in the step of S6, the operator performs the maintenance treatment for maintaining the size of the through-hole Hh. In the maintenance treatment, high-frequency energy is applied to an edge portion of the through-hole Hh through the energy transfer element 22, thereby cauterizing (heating and cauterizing) the edge portion of the through-hole Hh with the high-frequency energy.

The energy transfer element 22 in contact with the thick portion of the atrial septum HA is brought into firm contact in a similar manner to other energy transfer elements 22 as the second strut 64 corresponding to the energy transfer element 22 deforms. Thus, even when the thickness of the atrial septum HA is non-uniform in the circumferential direction, all the energy transfer elements 22 arranged in the recess 55 are appropriately brought into contact with the atrial septum HA. Therefore, according to the maintenance treatment, the entire edge portion of the through-hole Hh in the circumferential direction may be appropriately cauterized. In addition, the energy transfer elements 22 to which the current is supplied may be suppressed from being exposed to a blood vessel without being in contact with the biological tissue, whereby thrombus formation may be suppressed.

When the biological tissue in the vicinity of the edge portion of the through-hole Hh is cauterized through the energy transfer element 22, a degenerated portion in which the biological tissue is degenerated is formed in the vicinity of the edge portion. The biological tissue in the degenerated portion loses elasticity so that the through-hole Hh is enabled to maintain the shape enlarged by the expansion body 21.

The hemodynamics is checked again in the step of S7 after the maintenance treatment, and in a case where the blood volume passing through the through-hole Hh reaches desired volume, the operator decreases the diameter of the expansion body 21, stores the expansion body 21 in the outer tube 30, and removes it from the through-hole Hh. Moreover, the operator removes the entire medical device 10 from the living body to the outside of the living body, and terminates the treatment.

As described above, the medical device 10 according to the first embodiment includes: the expansion body 21 that has a distal end part including the force receiving portion 51 and is expandable/contractible in the radial direction; the elongated shaft portion 31 having a distal end part to which the proximal end of the expansion body 21 is fixed; the plurality of electrode portions (energy transfer elements 22) disposed along the expansion body 21; and the pulling shaft 33 that is disposed inside the shaft portion 31, connectable to the force receiving portion 51 of the expansion body 21 by protruding from the distal end part of the shaft portion 31, and slidable with respect to the shaft portion 31, in which the expansion body 21 includes: the first expansion portion 53 having the distal-side expansion portion 56 extending radially outward from the force receiving portion 51 toward the direction of the proximal end and the distal-side top portion 57 disposed on the proximal side of the distal-side expansion portion 56 and convexly curved radially outward; the second expansion portion 54 having the proximal-side expansion portion 58 extending radially outward from the distal end part of the shaft portion 31 toward the direction of the distal end and the proximal-side top portion 59 disposed on the distal side of the proximal-side expansion portion 58 and convexly curved radially outward; and the recess 55 that is recessed radially inward, extends to couple the proximal-side top portion 59 with the distal-side top portion 57, and defines the reception space 74 configured to receive a biological tissue when the expansion body 21 is expanded, the recess 55 has the bottom portion 71 located on the innermost side in the radial direction, the distal-side upright portion 72 extending radially outward from the distal end of the bottom portion 71 to the distal-side top portion 57, and the proximal-side upright portion 73 extending radially outward from the proximal end of the bottom portion 71 to the proximal-side top portion 59, one of the distal-side upright portion 72 or the proximal-side upright portion 73 includes the plurality of energy transfer element arrangement portions 81 on which the plurality of individual electrode portions is disposed at substantially regular intervals in the circumferential direction of the expansion body 21, the other one of the distal-side upright portion 72 or the proximal-side upright portion 73 includes the plurality of facing portions 82 facing the plurality of individual energy transfer elements 22 when the expansion body 21 is expanded, the distal-side expansion portion 56 includes the plurality of distal-side strut structures 60 coupled to the distal-side top portion 57, the proximal-side expansion portion 58 includes the plurality of proximal-side strut structures 90 coupled to the proximal-side top portion 59, at least one of the distal-side strut structures 60, the proximal-side strut structures 90, the energy transfer element arrangement portions 81, or the facing portions 82 have the easy-to-deform portions configured to be deformed more easily than other portions of the distal-side strut structures 60, the proximal-side strut structures 90, the energy transfer element arrangement portions 81, or the facing portions 82 when a force in the axial direction of the expansion body 21 is received, and each of the easy-to-deform portions deforms to enlarge the reception space 74 at the position corresponding to the easy-to-deform portion in the circumferential direction.

In the medical device 10 configured as described above, the easy-to-deform portion deforms when a force in the axial direction acts on the expansion body 21 so that the reception space 74 at a position in the circumferential direction corresponding to the easy-to-deform portion can be enlarged. Thus, by deforming the easy-to-deform portion, it becomes possible to appropriately bring the plurality of energy transfer elements 22, which is arranged in the recess 55 defining the reception space 74, into contact with the biological tissue having variations in thickness. Therefore, the medical device 10 may effectively cauterize the biological tissue having variations in thickness and may suppress thrombus formation.

Furthermore, the easy-to-deform portion has bending rigidity lower than that of other portions of the distal-side strut structure 60, the proximal-side strut structure 90, the energy transfer element arrangement portion 81, and the facing portion 82. With this arrangement, the force in the axial direction acts on the expansion body 21 and the easy-to-deform portion is bent, whereby the reception space 74 at the position in the circumferential direction corresponding to the easy-to-deform portion may be effectively enlarged.

Furthermore, the present disclosure also provides the method for forming a shunt. The method for forming a shunt is a method for forming a shunt that forms, in an oval fossa, a shunt (through-hole Hh) through which the right atrium HRa communicates with the left atrium HLa using the medical device 10 described above, the method including: inserting the medical device 10 from the inferior vena cava Iv into the right atrium HRa; inserting the expansion body 21 in the contracted state into the through-hole Hh formed in the oval fossa; expanding the expansion body 21 in the through-hole Hh to dispose the biological tissue surrounding the through-hole Hh in the reception space 74 defined by the recess 55; sliding the pulling shaft 33 in the direction of the proximal end with respect to the shaft portion 31 to compress the expansion body 21 such that the distal-side upright portion 72 and the proximal-side upright portion 73 of the recess 55 approach each other; changing, according to thickness of the biological tissue surrounding the through-hole Hh, a distance between the distal-side upright portion 72 and the proximal-side upright portion 73 in the circumferential direction of the expansion body 21 on the basis of deformation of the easy-to-deform portion to bring the energy transfer elements 22 disposed to face the recess 55 along the distal-side upright portion 72 or the proximal-side upright portion 73 of the recess 55 into contact with the biological tissue; and cauterizing the biological tissue disposed in the reception space 74 using the energy transfer elements 22 in contact with the biological tissue to inhibit occlusion due to natural healing of the through-hole Hh.

According to the method for forming a shunt configured as described above, the easy-to-deform portion deforms when the force in the axial direction of the expansion body 21 is received, thereby cauterizing the biological tissue disposed in the reception space 74 using the energy transfer element 22 in contact with the biological tissue having variations in thickness. The method for forming a shunt may effectively cauterize the biological tissue having variations in thickness and may suppress thrombus formation.

The present disclosure is not limited to the embodiment described above, and various modifications may be made by those skilled in the art within the technical idea of the present disclosure. For example, the position at which the easy-to-deform portion is disposed is not limited to the distal-side strut structure 60, and it may be disposed on the proximal-side strut structure 90, the energy transfer element arrangement portion 81, or the facing portion 82. Furthermore, the easy-to-deform portion may be disposed on two or more positions selected from the distal-side strut structure 60, the proximal-side strut structure 90, the energy transfer element arrangement portion 81, or the facing portion 82.

Furthermore, the proximal-side strut structure 90, the energy transfer element arrangement portion 81, the facing portion 82, and the distal-side strut structure 60 may be formed of one strut without branching and joining. Furthermore, the direction in which the easy-to-deform portion deforms is not particularly limited. Furthermore, the width of the strut may be partially changed so that the easy-to-deform portion is easily deformed when a predetermined force or more is applied.

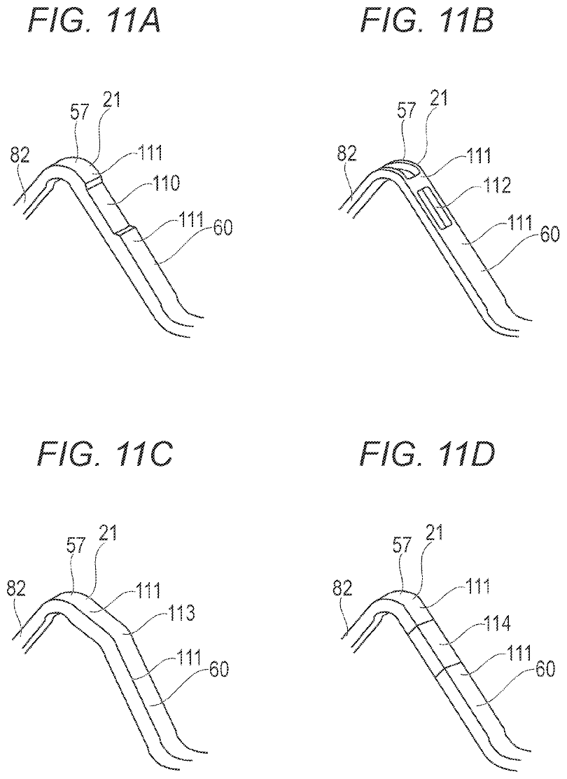

Furthermore, as in a first modified example of the first embodiment illustrated in FIG. 11A, the easy-to-deform portion may include a thin portion 110 whose thickness in the radial direction of the expansion body 21 is thinner than that of an adjacent portion of the expansion body 21. The thin portion 110 is a portion in which the second moment of area is smaller than that in the adjacent portion of the expansion body 21. With this arrangement, it becomes possible to rather easily set the easy-to-deform portion, which is easily bent, in the expansion body 21. Furthermore, it becomes possible to rather easily define the direction in which the easy-to-deform portion is bent. Examples of the method for forming the thin portion 110 include a method of reinforcing a portion other than the thin portion 110 of the expansion body 21 with metal or resin, a method of swaging by applying a pressing force, a method of scraping, and the like.

Furthermore, the easy-to-deform portion may be sandwiched between rigid portions 111 having bending rigidity higher than that of the easy-to-deform portion in the axial direction of the expansion body 21. With this arrangement, it becomes possible to concentrate the stress on the easy-to-deform portion when the force in the axial direction acts on the expansion body 21 so that the easy-to-deform portion may be easily bent.

Furthermore, as in a second modified example of the first embodiment illustrated in FIG. 11B, the easy-to-deform portion may have an opening 112 penetrating in the radial direction of the expansion body 21. With this arrangement, it becomes possible to rather easily set the easy-to-deform portion, which is easily bent, in the expansion body 21. Note that the opening that decreases the bending rigidity of the expansion body 21 may also be formed in the distal-side top portion 57, the proximal-side top portion 59, and the bottom portion 71.

Furthermore, as in a third modified example of the first embodiment illustrated in FIG. 11C, the easy-to-deform portion may have a bent portion 113 bent in a natural state. The direction in which the bent portion 113 is bent is not particularly limited, and can be, for example, a direction along the radial direction of the expansion body 21. With this arrangement, it becomes possible to concentrate the stress on the bent portion 113 when the force in the axial direction acts on the expansion body 21 so that the easy-to-deform portion may be easily bent. In a case where the easy-to-deform portion is easily deformed by being bent, the easy-to-deform portion does not necessarily have the bending rigidity lower than that of other portions of the distal-side strut structure 60, the proximal-side strut structure 90, the energy transfer element arrangement portion 81, and the facing portion 82.

Furthermore, as in a fourth modified example of the first embodiment illustrated in FIG. 11D, the easy-to-deform portion may include a flexible portion 114 made of a material more flexible than the material of the adjacent portion of the expansion body 21. For example, the flexible portion is made of resin, and the adjacent portion of the flexible portion 114 is made of metal, for example. With this arrangement, it becomes possible to easily set the easy-to-deform portion, which is easily bent, in the expansion body 21.

### Second Embodiment

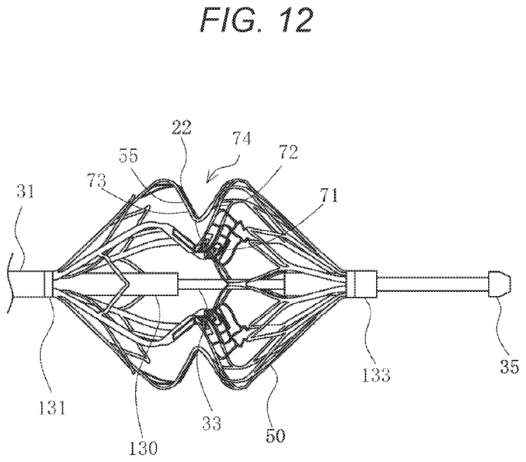

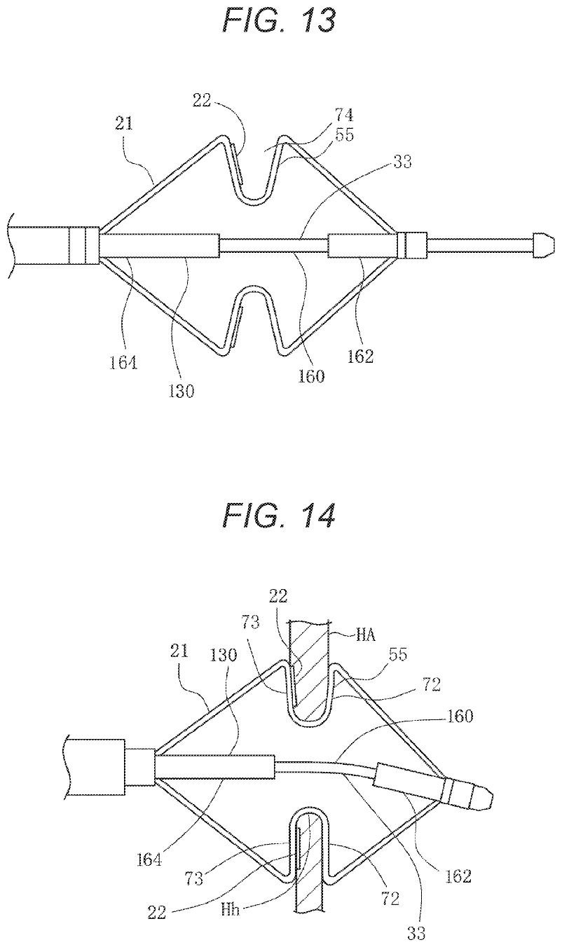

As illustrated in FIGS. 12 and 13, in a medical device 10 according to a second embodiment, a shaft portion 31 includes a distal-end shaft portion 130 including a proximal-end fixing portion 131 to which a proximal end of an expansion body 21 is fixed and a distal-end fixing portion 133 to which a distal end of the expansion body 21 is fixed. The distal-end shaft portion 130 extends inside the expansion body 21 from a proximal end part to a distal end part of the expansion body 21. Note that components common to those of the medical device 10 according to the first embodiment are denoted by the same reference numerals, and descriptions of the components common those of the medical device 10 will be omitted to avoid duplication.

The distal-end shaft portion 130 includes a flexible portion 160 configured to be bent at the center in the axial direction in a state where the expansion body 21 is expanded, a distal-end rigid portion 162 disposed on a side distal of the flexible portion 160 in the axial direction, and a proximal-end rigid portion 164 disposed on a side proximal of the flexible portion 160 in the axial direction. The distal-end rigid portion 162 and the proximal-end rigid portion 164 are formed of a hard outer pipe into which a pulling shaft 33 may be inserted. The flexible portion 160 is formed by a portion of the pulling shaft 33 exposed from the distal-end rigid portion 162 and the proximal-end rigid portion 164. Since the pulling shaft 33 is made of a bendable material, the flexible portion 160 may be bent by receiving a force. The distal-end rigid portion 162 and the proximal-end rigid portion 164 are made of a hard resin or metal to maintain a linear shape without being bent even when the flexible portion 160 receives a bending force.

Each of portions on which the proximal-end fixing portion 131 and the distal-end fixing portion 133 of the expansion body 21 are disposed is a binding portion at which a plurality of wire rod portions 50 converges, and the distal-end rigid portion 162 and the proximal-end rigid portion 164 extend toward the center in the axial direction from the proximal-end fixing portion 131 and the distal-end fixing portion 133, respectively. The distal-end rigid portion 162 and the proximal-end rigid portion 164 have a length of at least equal to or longer than 30% of the axial length of the portion in which the wire rod portion 50 extends from the binding portion toward a recess 55. In addition, the flexible portion 160 is disposed in a portion of the distal-end shaft portion 130 facing a bottom portion 71 of the recess 55 in the radial direction in the state where the expansion body 21 is expanded.

A treatment method using the medical device 10 according to the second embodiment is substantially similar to the treatment method using the medical device 10 according to the first embodiment. An operator grips an atrial septum HA with a proximal-side upright portion 73 and a distal-side upright portion 72, and presses an electrode portion (energy transfer element 22) against a biological tissue. In a case where the thickness of the biological tissue around a puncture hole Hh varies in a circumferential direction, as illustrated in FIG. 14, the flexible portion 160 of the distal-end shaft portion 130 is bent according to the thickness of the biological tissue as the expansion body 21 is compressed by the pulling shaft 33. As a result, the recess 55 of the expansion body 21 is brought into contact with the biological tissue over the entire circumference in the circumferential direction. Therefore, it becomes possible to reliably bring the electrode portion (energy transfer element 22) into contact with the biological tissue. In FIG. 14, the thickness of the biological tissue on the upper side in the drawing of the puncture hole Hh is larger, and the thickness of the biological tissue on the lower side in the drawing of the puncture hole Hh is smaller. In the distal-end shaft portion 130, the flexible portion 160 is bent downward in the drawing according to the difference in thickness of the biological tissue. Accordingly, in the recess 55 of the expansion body 21 on the upper side in the drawing, the interval between the proximal-side upright portion 73 and the distal-side upright portion 72 is wider according to the larger thickness of the biological tissue, and in the recess 55 of the expansion body 21 on the lower side in the drawing, the interval between the proximal-side upright portion 73 and the distal-side upright portion 72 is narrower according to the smaller thickness of the biological tissue. Thus, both of the portions of the biological tissue having larger and smaller thicknesses are gripped by the recess 55 with the equivalent force, and the individual electrode portions (energy transfer element 22) are brought into contact with the biological tissue with the equivalent force.

Since the distal-end shaft portion 130 includes the distal-end rigid portion 162 and the proximal-end rigid portion 164 and includes the bendable flexible portion 160 in the portion radially facing the bottom portion 71 of the recess 55, it becomes possible to form a bent shape at the center of the distal-end shaft portion 130 in the axial direction. As a result, the expansion body 21 may be deformed such that the proximal-side upright portion 73 and the distal-side upright portion 72 approach each other depending on the thickness of the biological tissue along the circumferential direction of the recess 55. While the bent shape at the center of the distal-end shaft portion 130 in the axial direction may not be formed when the distal-end shaft portion 130 is entirely formed of the flexible portion 160 so that the recess 55 fails to grip the biological tissue at least in a part in the circumferential direction, the bent shape at the center of the distal-end shaft portion 130 in the axial direction is achieved by the distal-end rigid portion 162 and the proximal-end rigid portion 164 being included in the distal-end shaft portion 130, whereby the expansion body 21 may be deformed such that the recess 55 grips the biological tissue over the entire circumference. In order to form such a bent shape at the center of the distal-end shaft portion 130 in the axial direction, the distal-end rigid portion 162 and the proximal-end rigid portion 164 need to have a certain length. Accordingly, as described above, the distal-end rigid portion 162 and the proximal-end rigid portion 164 have a length of at least equal to or longer than 30% of the axial length of the portion in which the wire rod portion 50 extends from the binding portion toward the recess 55.

Next, the operator checks hemodynamics (S5), inhibits occlusion due to natural healing of the puncture hole Hh, and performs a maintenance treatment to maintain the size thereof (S6). In the maintenance treatment, high-frequency energy is applied to an edge portion of the puncture hole Hh through the electrode portion (energy transfer element 22), thereby cauterizing (heating and cauterizing) the edge portion of the puncture hole Hh with the high-frequency energy. The high-frequency energy is applied by a voltage being applied between a pair of electrode portions (energy transfer elements 22) adjacent in the circumferential direction. As described above, since the distal-end shaft portion 130 is bent at the center in the axial direction so that the individual electrode portions (energy transfer elements 22) are uniformly brought into contact with the biological tissue, it becomes possible to reliably apply the energy to the biological tissue over the entire circumference by applying a voltage to the electrode portions (energy transfer elements 22) even when the thickness of the biological tissue surrounding the puncture hole Hh is different in the circumferential direction.

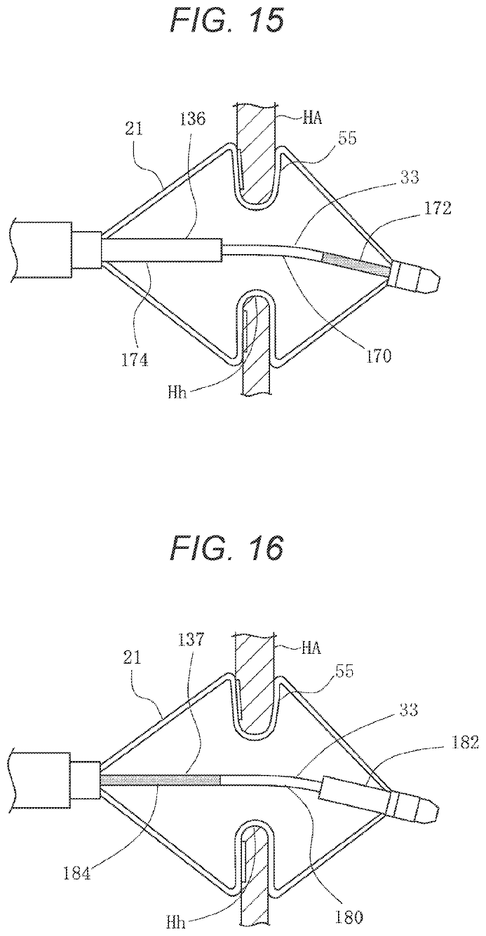

Next, a modified example of the distal-end shaft portion in the second embodiment will be described. As illustrated in FIG. 15, a distal-end shaft portion 136 according to a fifth modified example in the second embodiment includes a flexible portion 170 in an intermediate portion in the axial direction, a distal-end rigid portion 172 on the side distal of the flexible portion 170, and a proximal-end rigid portion 174 on the side proximal of the flexible portion 170. The proximal-end rigid portion 174 is formed of a hard outer pipe into which the pulling shaft 33 is inserted. The pulling shaft 33 includes the flexible portion 170 exposed to the side distal of the proximal-end rigid portion 174 in the axial direction, and the distal-end rigid portion 172 disposed on the side distal of the flexible portion 170 in the axial direction. That is, the distal-end rigid portion 172 is formed on the pulling shaft 33. The distal-end rigid portion 172 may be formed such that, for example, the surface of the flexibly formed pulling shaft 33 is coated with a hard tubular member. As described above, even when the distal-end rigid portion 172 is formed on the pulling shaft 33, the flexible portion 170 of the distal-end shaft portion 136 is bent so that the expansion body 21 may deform to have a shape of the recess 55 according to the thickness of the biological tissue along the circumferential direction in the case where the thickness of the biological tissue around the puncture hole Hh varies in the circumferential direction.

As illustrated in FIG. 16, a distal-end shaft portion 137 according to a sixth modified example in the second embodiment includes a flexible portion 180 in an intermediate portion in the axial direction, a distal-end rigid portion 182 on the side distal of the flexible portion 180, and a proximal-end rigid portion 184 on the side proximal of the flexible portion 180. The distal-end rigid portion 182 is formed of a hard outer pipe into which the pulling shaft 33 is inserted. The pulling shaft 33 includes the flexible portion 180 exposed to the side proximal of the distal-end rigid portion 182 in the axial direction, and the proximal-end rigid portion 184 disposed on the side proximal of the flexible portion 180 in the axial direction. That is, the proximal-end rigid portion 184 is formed on the pulling shaft 33. As described above, the proximal-end rigid portion 184 may be formed on the pulling shaft 33.

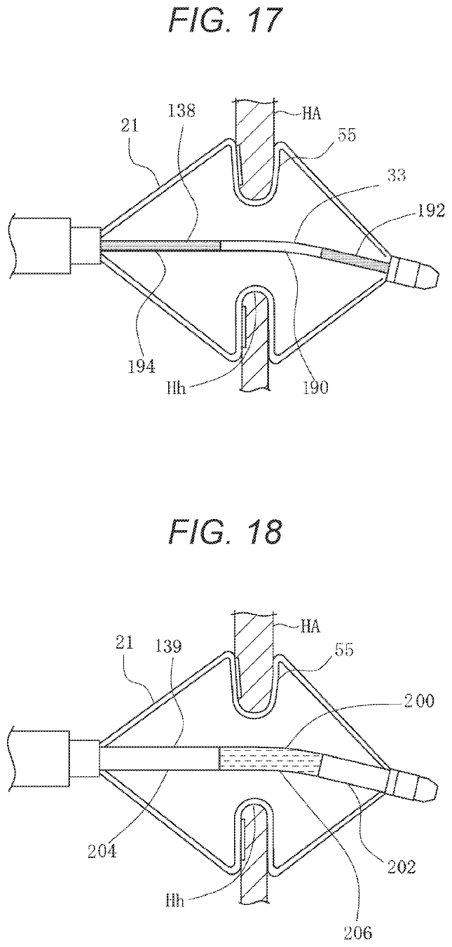

As illustrated in FIG. 17, a distal-end shaft portion 138 according to a seventh modified example in the second embodiment includes a flexible portion 190 in an intermediate portion in the axial direction, a distal-end rigid portion 192 on the side distal of the flexible portion 190, and a proximal-end rigid portion 194 on the side proximal of the flexible portion 190. Both of the distal-end rigid portion 192 and the proximal-end rigid portion 194 are formed on the pulling shaft 33, and a portion between the distal-end rigid portion 192 and the proximal-end rigid portion 194 is the flexible portion 190. In this manner, both of the distal-end rigid portion 192 and the proximal-end rigid portion 194 may be formed on the pulling shaft 33.

As illustrated in FIG. 18, a distal-end shaft portion 139 according to an eighth modified example in the second embodiment includes a flexible portion 200 in an intermediate portion in the axial direction, a distal-end rigid portion 202 on the side distal of the flexible portion 200, and a proximal-end rigid portion 204 on the side proximal of the flexible portion 200. The flexible portion 200, the distal-end rigid portion 202, and the proximal-end rigid portion 204 are all formed on an outer pipe 206 into which the pulling shaft 33 is inserted. The outer pipe 206 is made of a relatively hard material, and the portion of the flexible portion 200 is made of a relatively flexible material. Alternatively, the outer pipe 206 may be entirely made of a relatively hard material, and a large number of slits or holes may be formed in the portion of the flexible portion 200 to form the flexible portion 200 that is easily bent. In this manner, the flexible portion 200, the distal-end rigid portion 202, and the proximal-end rigid portion 204 may all be formed on the outer pipe 206.

As described above, the medical device 10 according to the second embodiment includes: the expansion body 21 that is expandable/contractible in the radial direction; the elongated shaft portion 20 including, in the distal end part, the proximal-end fixing portion 131 to which the proximal end of the expansion body 21 is fixed; the pulling shaft 33 that is disposed inside the shaft portion 20, connected to the distal end part of the expansion body 21 by protruding from the distal end part of the shaft portion 20, and slidable with respect to the shaft portion 20; the distal-end shaft portion 130 that extends inside the expansion body 21 from the proximal end part to the distal end part of the expansion body 21; and the electrode portion 22 disposed along the expansion body 21, in which the expansion body 21 includes the recess 55 that is recessed radially inward and defines the reception space 74 configured to receive a biological tissue when the expansion body 21 is expanded, the recess 55 includes the bottom portion 71 located on the innermost side in the radial direction, the distal-side upright portion 72 extending radially outward from the distal end of the bottom portion 71, and the proximal-side upright portion 73 extending radially outward from the proximal end of the bottom portion 71, the electrode portion (energy transfer element 22) is disposed along the distal-side upright portion 72 or the proximal-side upright portion 73 to face the reception space 74, the pulling shaft 33 is configured to apply, to the expansion body 21, a compressive force that makes compression along the axial center of the shaft portion 20 such that the distal-side upright portion 72 and the proximal-side upright portion 73 approach each other by sliding in the direction of the proximal end with respect to the shaft portion 20, and in a state where the expansion body 21 is expanded, the distal-end shaft portion 130 includes the flexible portion 160 configured to be bent at the center in the axial direction, the distal-end rigid portion 162 disposed on the side distal of the flexible portion 160 in the axial direction, and the proximal-end rigid portion 164 disposed on the side proximal of the flexible portion 160 in the axial direction.

Furthermore, the method for forming a shunt according to the second embodiment forms, in an oval fossa, a shunt through which a right atrium communicates with a left atrium using the medical device 10 including the expansion body 21 that is expandable/contractible in the radial direction, the elongated shaft portion 20 including, in the distal end part, the proximal-end fixing portion 131 to which the proximal end of the expansion body 21 is fixed, the pulling shaft 33 that is disposed inside the shaft portion 20, connected to the distal end part of the expansion body 21 by protruding from the distal end part of the shaft portion 20, and slidable with respect to the shaft portion 20, the distal-end shaft portion 130 that extends inside the expansion body 21 from the proximal end part to the distal end part of the expansion body 21, and the electrode portion 22 disposed along the expansion body 21, in which in a state where the expansion body 21 is expanded, the distal-end shaft portion 130 includes the flexible portion 160 configured to be bent at the center in the axial direction, the distal-end rigid portion 162 disposed on the side distal of the flexible portion 160 in the axial direction, and the proximal-end rigid portion 164 disposed on the side proximal of the flexible portion 160 in the axial direction, the method including: inserting the medical device 10 from an inferior vena cava into the right atrium; inserting the expansion body 21 in the contracted state into a hole formed in the oval fossa; expanding the expansion body 21 in the hole to dispose a biological tissue surrounding the hole in the reception space 74 defined by the recess 55 of the expansion body 21 including the bottom portion 71 located on the innermost side in the radial direction, the distal-side upright portion 72 extending radially outward from the distal end of the bottom portion 71, and the proximal-side upright portion 73 extending radially outward from the proximal end of the bottom portion 71; compressing, by sliding the pulling shaft 33 in the direction of the proximal end with respect to the shaft portion 20, the expansion body 21 such that the distal-side upright portion 72 and the proximal-side upright portion 73 of the recess 55 approach each other to bend the flexible portion 160 according to thickness of the biological tissue surrounding the hole; bringing the electrode portion 22 disposed to face the recess 55 along the distal-side upright portion 72 or the proximal-side upright portion 73 of the recess 55 into contact with the biological tissue by bending of the flexible portion 160; and cauterizing the biological tissue disposed in the reception space 74 using the electrode portion 22 in contact with the biological tissue to inhibit occlusion due to natural healing of the hole.

According to the medical device 10 and the method for forming a shunt according to the second embodiment configured as described above, it becomes possible to, when the thickness of the biological tissue to be in contact with the expansion body 21 varies along the circumferential direction, bend the distal-end shaft portion 130 at the portion of the flexible portion 160 depending on the thickness of the biological tissue to deform the expansion body 21 such that the recess 55 is brought into contact with each of portions of the biological tissue having larger and smaller thicknesses. As a result, it becomes possible to reliably bring the electrode portion (energy transfer element 22) into contact with the biological tissue over the entire circumference.

The distal-end rigid portion 162 and the proximal-end rigid portion 164 may be formed of an outer pipe into which the pulling shaft 33 is inserted, and the flexible portion 160 may be formed of a portion of the pulling shaft 33 exposed from the distal-end rigid portion 162 and the proximal-end rigid portion 164. With this arrangement, the rigidity of the distal-end rigid portion 162 and the proximal-end rigid portion 164 may be sufficiently secured.

The proximal-end rigid portion 174 may be formed of an outer pipe into which the pulling shaft 33 is inserted, and the pulling shaft 33 may include the flexible portion 170 exposed to the side distal of the proximal-end rigid portion 174 in the axial direction, and the distal-end rigid portion 172 disposed on the side distal of the flexible portion 170 in the axial direction. With this arrangement, it becomes possible to reduce the number of outer pipes to facilitate assembly.

The distal-end rigid portion 182 may be formed of an outer pipe into which the pulling shaft 33 is inserted, and the pulling shaft 33 may include the flexible portion 180 exposed to the side proximal of the distal-end rigid portion 182 in the axial direction, and the proximal-end rigid portion 184 disposed on the side proximal of the flexible portion 180 in the axial direction. With this arrangement, it becomes possible to reduce the number of outer pipes to facilitate assembly.

The distal-end shaft portion 139 may be formed of an outer pipe into which the pulling shaft 33 is inserted, and the distal-end shaft portion 139 may include the flexible portion 200, the distal-end rigid portion 202, and the proximal-end rigid portion 204. With this arrangement, it becomes possible to reduce the number of outer pipes and eliminate the need to process the pulling shaft 33.

The pulling shaft 33 may include the flexible portion 190, the distal-end rigid portion 192, and the proximal-end rigid portion 194. With this arrangement, it becomes possible to form the distal-end rigid portion 192 and the proximal-end rigid portion 194 only with the pulling shaft 33, whereby the number of parts may be further reduced.

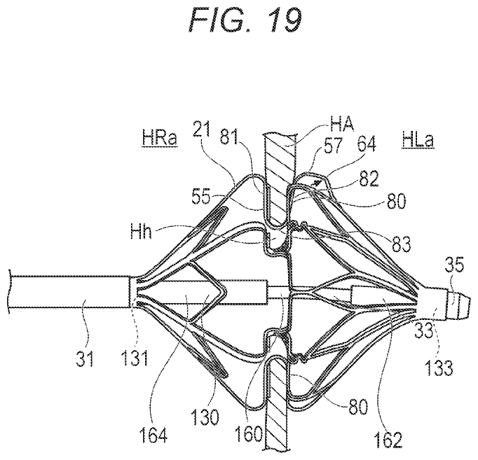

Furthermore, the medical device 10 according to the first embodiment may include the distal-end shaft portions 130, 136, 137, 138, and 139 according to the second embodiment. For example, as in a ninth modified example illustrated in FIG. 19, the medical device 10 according to the first embodiment includes the distal-end shaft portion 130 according to the second embodiment. The distal-end shaft portion 130 includes the proximal-end fixing portion 131 to which the proximal end of the expansion body 21 is fixed, and the distal-end fixing portion 133 to which the distal end of the expansion body 21 is fixed. The distal-end shaft portion 130 includes the flexible portion 160, the distal-end rigid portion 162 disposed on the side distal of the flexible portion 160 in the axial direction, and the proximal-end rigid portion 164 disposed on the side proximal of the flexible portion 160 in the axial direction. With this arrangement, according to the medical device 10 and the method for forming a shunt in the ninth modified example, when the thickness of the biological tissue to be in contact with the expansion body 21 varies along the circumferential direction, the easy-to-deform portion (second strut 64) deforms so that the reception space 74 at the position corresponding to the easy-to-deform portion in the circumferential direction increases and the distal-end shaft portion 130 is bent at the position of the flexible portion 160 depending on the thickness of the biological tissue, whereby the expansion body 21 may deform such that the recess 55 is brought into contact with each of portions of the biological tissue having larger and smaller thicknesses. With this arrangement, it becomes possible to more reliably bring the electrode portion 22 into contact with the biological tissue over the entire circumference.

The detailed description above describes embodiments of a medical device including an expansion body that expands in a living body and a method for forming a shunt. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A medical device comprising:
an expansion body that includes a distal end part including a force receiving portion and is configured to be expandable and contractible in a radial direction;
an elongated shaft portion including a distal end part to which a proximal end of the expansion body is fixed;
a plurality of energy transfer elements disposed along the expansion body;
a pulling shaft that is disposed inside the shaft portion, connectable to the force receiving portion of the expansion body by protruding from the distal end part of the shaft portion, and slidable with respect to the shaft portion; and
wherein the expansion body includes:
a first expansion portion including a distal-side expansion portion extending radially outward from the force receiving portion toward a direction of the proximal end and a distal-side top portion disposed on a proximal side of the distal-side expansion portion and convexly curved radially outward;
a second expansion portion including a proximal-side expansion portion extending radially outward from the distal end part of the shaft portion toward a direction of the distal end and a proximal-side top portion disposed on a distal side of the proximal-side expansion portion and convexly curved radially outward;
a recess that is recessed radially inward, extends to couple the proximal-side top portion with the distal-side top portion, and defines a reception space that can receive a biological tissue when the expansion body is expanded;
the recess includes a bottom portion located on an innermost side in the radial direction, a distal-side upright portion extending radially outward from a distal end of the bottom portion to the distal-side top portion, and a proximal-side upright portion extending radially outward from a proximal end of the bottom portion to the proximal-side top portion;
one of the distal-side upright portion or the proximal-side upright portion includes a plurality of energy transfer element arrangement portions on which the plurality of individual energy transfer elements is disposed at a substantially regular interval in a circumferential direction of the expansion body;
another one of the distal-side upright portion or the proximal-side upright portion includes a plurality of facing portions facing the plurality of individual energy transfer elements when the expansion body is expanded;
the distal-side expansion portion includes a plurality of distal-side strut structures coupled to the distal-side top portion;
the proximal-side expansion portion includes a plurality of proximal-side strut structures coupled to the proximal-side top portion;
at least one of the distal-side strut structures, the proximal-side strut structures, the energy transfer element arrangement portions, or the facing portions include easy-to-deform portions that can be deformed more easily than other portions of the distal-side strut structures, the proximal-side strut structures, the energy transfer element arrangement portions, or the facing portions when a force in an axial direction of the expansion body is received; and
each of the easy-to-deform portions deforms to enlarge the reception space at a position corresponding to the easy-to-deform portion in the circumferential direction.

2. The medical device according to claim 1, wherein the easy-to-deform portions include bending rigidity lower than the bending rigidity of the other portions of the distal-side strut structures, the proximal-side strut structures, the energy transfer element arrangement portions, or the facing portions.

3. The medical device according to claim 2, wherein the easy-to-deform portion includes an opening that penetrates in the radial direction of the expansion body.

4. The medical device according to claim 2, wherein the easy-to-deform portion includes a thin portion with thickness in the radial direction of the expansion body smaller than thickness of an adjacent portion of the expansion body.

5. The medical device according to claim 2, wherein the easy-to-deform portion includes a flexible portion comprised of a material more flexible than a material of an adjacent portion of the expansion body.

6. The medical device according to claim 2, wherein the easy-to-deform portion is sandwiched between rigid portions including the bending rigidity higher than the bending rigidity of the easy-to-deform portion in the axial direction of the expansion body.

7. The medical device according to claim 1, wherein the easy-to-deform portion includes a bent portion bent in a natural state.

8. The medical device according to claim 1, further comprising:
a distal-end shaft portion that extends inside the expansion body from a proximal end part to the distal end part of the expansion body;
each of the plurality of energy transfer elements is comprised of an electrode portion;
the pulling shaft is configured to apply, to the expansion body, a compressive force configured to cause a compression along an axial center of the shaft portion such that the distal-side upright portion and the proximal-side upright portion approach each other by sliding in the direction of the proximal end with respect to the shaft portion; and
in a state in which the expansion body is expanded, the distal-end shaft portion includes a flexible portion bendable at a center in the axial direction, a distal-end rigid portion disposed on a side distal of the flexible portion in the axial direction, and a proximal-end rigid portion disposed on a side proximal of the flexible portion in the axial direction.

9. A method for forming a shunt that forms, in an oval fossa, a shunt through which a right atrium communicates with a left atrium using a medical device including an expansion body that includes a distal end part including a force receiving portion and is expandable/contractible in a radial direction, an elongated shaft portion including a distal end part to which a proximal end of the expansion body is fixed, a plurality of energy transfer elements disposed along the expansion body, and a pulling shaft that is disposed inside the shaft portion, connectable to the force receiving portion of the expansion body by protruding from the distal end part of the shaft portion, and slidable with respect to the shaft portion, in which the expansion body includes a first expansion portion including a distal-side expansion portion extending radially outward from the force receiving portion toward a direction of the proximal end and a distal-side top portion disposed on a proximal side of the distal-side expansion portion and convexly curved radially outward, a second expansion portion including a proximal-side expansion portion extending radially outward from the distal end part of the shaft portion toward a direction of the distal end and a proximal-side top portion disposed on a distal side of the proximal-side expansion portion and convexly curved radially outward, and a recess that is recessed radially inward, extends to couple the proximal-side top portion with the distal-side top portion, and defines a reception space that can receive a biological tissue when the expansion body is expanded, the recess includes a bottom portion located on an innermost side in the radial direction, a distal-side upright portion extending radially outward from a distal end of the bottom portion to the distal-side top portion, and a proximal-side upright portion extending radially outward from a proximal end of the bottom portion to the proximal-side top portion, one of the distal-side upright portion or the proximal-side upright portion includes a plurality of energy transfer element arrangement portions on which the plurality of individual energy transfer elements is disposed at a substantially regular interval in a circumferential direction of the expansion body, another one of the distal-side upright portion or the proximal-side upright portion includes a plurality of facing portions facing the plurality of individual energy transfer elements when the expansion body is expanded, the distal-side expansion portion includes a plurality of distal-side strut structures coupled to the distal-side top portion, the proximal-side expansion portion includes a plurality of proximal-side strut structures coupled to the proximal-side top portion, and at least one of the distal-side strut structures, the proximal-side strut structures, the energy transfer element arrangement portions, or the facing portions include easy-to-deform portions that can be deformed more easily than other portions of the distal-side strut structures, the proximal-side strut structures, the energy transfer element arrangement portions, or the facing portions when a force in an axial direction of the expansion body is received, the method comprising:
inserting the medical device from an inferior vena cava into the right atrium;
inserting the expansion body in a contracted state into a hole formed in the oval fossa;
expanding the expansion body in the hole to dispose the biological tissue surrounding the hole in the reception space defined by the recess;
sliding the pulling shaft in the direction of the proximal end with respect to the shaft portion to compress the expansion body such that the distal-side upright portion and the proximal-side upright portion of the recess approach each other;
changing, according to thickness of the biological tissue surrounding the hole, a distance between the distal-side upright portion and the proximal-side upright portion in the circumferential direction of the expansion body on a basis of deformation of the easy-to-deform portion to bring the energy transfer elements disposed to face the recess along the distal-side upright portion or the proximal-side upright portion of the recess into contact with the biological tissue; and
cauterizing the biological tissue disposed in the reception space using the energy transfer elements in contact with the biological tissue to inhibit occlusion due to natural healing of the hole.

10. The method according to claim 9, wherein the easy-to-deform portions include bending rigidity lower than the bending rigidity of the other portions of the distal-side strut structures, the proximal-side strut structures, the energy transfer element arrangement portions, or the facing portions.

11. The method according to claim 10, wherein the easy-to-deform portion includes an opening that penetrates in the radial direction of the expansion body.

12. The method according to claim 10, wherein the easy-to-deform portion includes a thin portion with thickness in the radial direction of the expansion body smaller than thickness of an adjacent portion of the expansion body.

13. The method according to claim 10, wherein the easy-to-deform portion includes a flexible portion comprised of a material more flexible than a material of an adjacent portion of the expansion body.

14. The method according to claim 10, wherein the easy-to-deform portion is sandwiched between rigid portions including the bending rigidity higher than the bending rigidity of the easy-to-deform portion in the axial direction of the expansion body.

15. The method according to claim 9, wherein the easy-to-deform portion includes a bent portion bent in a natural state.

16. The method according to claim 9, further comprising:
a distal-end shaft portion that extends inside the expansion body from a proximal end part to the distal end part of the expansion body;
each of the plurality of energy transfer elements is comprised of an electrode portion;
the pulling shaft is configured to apply, to the expansion body, a compressive force configured to cause a compression along an axial center of the shaft portion such that the distal-side upright portion and the proximal-side upright portion approach each other by sliding in the direction of the proximal end with respect to the shaft portion; and
in a state in which the expansion body is expanded, the distal-end shaft portion includes a flexible portion bendable at a center in the axial direction, a distal-end rigid portion disposed on a side distal of the flexible portion in the axial direction, and a proximal-end rigid portion disposed on a side proximal of the flexible portion in the axial direction.

17. A medical device comprising:
an expansion body configured to be expandable and contractible in a radial direction;
an elongated shaft portion including a proximal-end fixing portion to which a proximal end of the expansion body is fixed, the proximal-end fixing portion being in a distal end part of the elongated shaft portion;
a pulling shaft that is disposed inside the shaft portion, connected to a distal end part of the expansion body by protruding from the distal end part of the shaft portion, and configured to be slidable with respect to the shaft portion;
a distal-end shaft portion that extends inside the expansion body from a proximal end part to the distal end part of the expansion body;
an electrode portion disposed along the expansion body, wherein the expansion body includes a recess that is recessed radially inward and defines a reception space that can receive a biological tissue when the expansion body is expanded;
the recess includes a bottom portion located on an innermost side in the radial direction, a distal-side upright portion extending radially outward from a distal end of the bottom portion, and a proximal-side upright portion extending radially outward from a proximal end of the bottom portion;
the electrode portion is disposed along the distal-side upright portion or the proximal-side upright portion to face the reception space;
the pulling shaft is configured to apply, to the expansion body, a compressive force that makes compression along an axial center of the shaft portion such that the distal-side upright portion and the proximal-side upright portion approach each other by sliding in a direction of the proximal end with respect to the shaft portion; and
in a state in which the expansion body is expanded, the distal-end shaft portion includes a flexible portion bendable at a center in an axial direction, a distal-end rigid portion disposed on a side distal of the flexible portion in the axial direction, and a proximal-end rigid portion disposed on a side proximal of the flexible portion in the axial direction.

18. The medical device according to claim 17, wherein the flexible portion is formed by a portion of the pulling shaft exposed from the distal-end rigid portion and the proximal-end rigid portion.

19. The medical device according to claim 17, wherein the distal-end rigid portion and the proximal-end rigid portion have a length of at least equal to or longer than 30% of the axial length of the portion in which the wire rod portion extends from the binding portion toward the recess.

20. A method for forming a shunt that forms, in an oval fossa, a shunt through which a right atrium communicates with a left atrium using the medical device of claim 17, the method comprising:
inserting the medical device from an inferior vena cava into the right atrium;
inserting the expansion body in a contracted state into a hole formed in the oval fossa;
expanding the expansion body in the hole to dispose a biological tissue surrounding the hole in a reception space defined by a recess of the expansion body including a bottom portion located on an innermost side in the radial direction, a distal-side upright portion extending radially outward from a distal end of the bottom portion, and a proximal-side upright portion extending radially outward from a proximal end of the bottom portion;
compressing, by sliding the pulling shaft in a direction of the proximal end with respect to the shaft portion, the expansion body such that the distal-side upright portion and the proximal-side upright portion of the recess approach each other to bend the flexible portion according to thickness of the biological tissue surrounding the hole;
bringing the electrode portion disposed to face the recess along the distal-side upright portion or the proximal-side upright portion of the recess into close contact with the biological tissue by bending of the flexible portion; and
cauterizing the biological tissue disposed in the reception space using the electrode portion in close contact with the biological tissue to inhibit occlusion due to natural healing of the hole.
